(19) United States
(12) Patent Application Publication
(10) Pub. No.: US 2023/0020046 A1
(43) Pub. Date: Jan. 19, 2023

(54) APPARATUS FOR COMPRESSING A REPLACEMENT HEART VALVE IMPLANT

(71) Applicant: BOSTON SCIENTIFIC SCIMED, INC., MAPLE GROVE, MN (US)

(72) Inventors: Declan Loughnane, Galway (IE); Tim O'Connor, Galway (IE); John Lardner, Galway (IE)

(73) Assignee: BOSTON SCIENTIFIC SCIMED, INC., MAPLE GROVE, MN (US)

(21) Appl. No.: 17/862,664

(22) Filed: Jul. 12, 2022

Related U.S. Application Data
(60) Provisional application No. 63/221,121, filed on Jul. 13, 2021.

(51) Int. Cl. A61F 2/95; A61F 2/24

(52) U.S. Cl. CPC A61F 2/9525; A61F 2/2418; A61F 2/2427

(57) ABSTRACT

An apparatus for compressing a replacement heart valve implant may include a tubular member having a wall having an outer surface and an inner surface defining a lumen of the tubular member, wherein the inner surface includes a tapered portion having a radially inward taper in a distal direction, and a plurality of flexible arms projecting radially inward from the tapered portion. The plurality of flexible arms may project radially inward from the inner surface at least 50% of a distance from the inner surface to a central longitudinal axis of the tubular member in an unbiased configuration. The plurality of flexible arms may be configured to push a plurality of leaflets of the replacement heart valve implant radially inward as the replacement heart valve implant passes through the tubular member.

### CROSS-REFERENCE TO RELATED APPLICATIONS

This application claims the benefit of priority of U.S. Provisional Application No. 63/221,121 filed Jul. 13, 2021, the entire disclosure of which is hereby incorporated by reference.

### TECHNICAL FIELD

The present disclosure pertains to medical devices, systems, and methods for manufacturing and/or using medical devices and/or systems. More particularly, the present disclosure pertains to an improved design for an apparatus for compressing a replacement heart valve implant and a method of using the apparatus.

### BACKGROUND

A wide variety of intracorporeal medical devices have been developed for medical use, for example, intravascular use. Some of these devices include guidewires, catheters, medical device delivery systems (e.g., for stents, grafts, replacement valves, etc.), and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### SUMMARY

In one example, an apparatus for compressing a replacement heart valve implant may comprise a tubular member having a wall having an outer surface and an inner surface defining a lumen of the tubular member, wherein the inner surface includes a tapered portion having a radially inward taper in a distal direction, and a plurality of flexible arms projecting radially inward from the tapered portion. The plurality of flexible arms projects radially inward from the inner surface at least 50% of a distance from the inner surface to a central longitudinal axis of the tubular member in an unbiased configuration.

In addition or alternatively to any example described herein, the plurality of flexible arms projects radially inward from the inner surface at least 75% of the distance from the inner surface to the central longitudinal axis of the tubular member in the unbiased configuration.

In addition or alternatively to any example described herein, the plurality of flexible arms is fixedly attached to the wall of the tubular member.

In addition or alternatively to any example described herein, the plurality of flexible arms is formed from a monofilament polymeric material.

In addition or alternatively to any example described herein, the plurality of flexible arms is formed from a different material then the tubular member.

In addition or alternatively to any example described herein, the apparatus may further comprise a threaded nut configured to engage threads formed in the outer surface of the tubular member, and a push member comprising an annular ring and a plurality of extension members extending radially inward from the annular ring. The annular ring is configured to slide over the outer surface of the tubular member.

In addition or alternatively to any example described herein, the tubular member includes a plurality of longitudinal slots extending through the wall. The plurality of extension members extends through the plurality of longitudinal slots into the lumen of the tubular member as the annular ring slides over the outer surface of the tubular member.

In addition or alternatively to any example described herein, a replacement heart valve system may comprise a replacement heart valve implant including an expandable framework and a plurality of leaflets secured to the expandable framework, and an apparatus for compressing the replacement heart valve implant. The apparatus may comprise a tubular member having a wall having an outer surface and an inner surface defining a lumen of the tubular member, wherein the inner surface includes a tapered portion having a radially inward taper in a distal direction, and a plurality of flexible arms projecting radially inward from the tapered portion. The plurality of flexible arms is configured to push the plurality of leaflets radially inward as the replacement heart valve implant passes through the tubular member.

In addition or alternatively to any example described herein, the plurality of flexible arms is equally spaced apart circumferentially around a central longitudinal axis of the tubular member.

In addition or alternatively to any example described herein, the plurality of flexible arms is spaced apart about 120 degrees from each other.

In addition or alternatively to any example described herein, each of the plurality of leaflets is engaged by only one of the plurality of flexible arms as the replacement heart valve implant passes through the tubular member.

In addition or alternatively to any example described herein, the plurality of leaflets is secured to the expandable framework at a plurality of commissures and includes free edges extending between the plurality of commissures, and wherein between circumferentially adjacent commissures the replacement heart valve implant is devoid of the expandable framework at a longitudinal position radially outward of the free edges of the plurality of leaflets.

In addition or alternatively to any example described herein, a method of compressing a replacement heart valve implant may comprise:

inserting an inflow end of the replacement heart valve implant into an apparatus for compressing the replacement heart valve implant, wherein the apparatus comprises:
a tubular member having a wall having an outer surface and an inner surface defining a lumen of the tubular member, wherein the inner surface includes a tapered portion having a radially inward taper in a distal direction; and
a plurality of flexible arms projecting radially inward from the tapered portion;

sliding a push member over a proximal end of the tubular member, the push member comprising an annular ring and a plurality of extension members extending radially inward from the annular ring, until the plurality of extension members engages a plurality of commissures of the replacement heart valve implant;

placing a threaded nut over the proximal end of the tubular member, the threaded nut being configured to engage threads formed in the outer surface of the tubular member;

advancing the threaded nut distally along the tubular member to translate the push member distally along the tubular member, the push member advancing the replacement heart valve implant distally within the lumen of the tubular member.

In addition or alternatively to any example described herein, as the replacement heart valve implant advances past the plurality of flexible arms, the plurality of flexible arms pushes a plurality of leaflets of the replacement heart valve implant radially inward from an expandable framework of the replacement heart valve implant.

In addition or alternatively to any example described herein, each of the plurality of flexible arms is disposed circumferentially between two circumferentially adjacent extension members of the plurality of the extension members.

In addition or alternatively to any example described herein, the plurality of extension members extends radially inward through a plurality of longitudinal slots formed in the tubular member.

In addition or alternatively to any example described herein, the plurality of longitudinal slots extends from the proximal end of the tubular member toward a distal end of the tubular member.

In addition or alternatively to any example described herein, each of the plurality of flexible arms is disposed circumferentially between two circumferentially adjacent longitudinal slots of the plurality of the longitudinal slots.

In addition or alternatively to any example described herein, advancing the replacement heart valve implant distally within the lumen of the tubular member radially compresses the replacement heart valve implant.

In addition or alternatively to any example described herein, the plurality of flexible arms extends from the inner surface of the tubular member in the distal direction within the lumen of the tubular member.

The above summary of some embodiments, aspects, and/or examples is not intended to describe each disclosed embodiment or every implementation of the present disclosure. The figures and detailed description which follow more particularly exemplify these embodiments.

### BRIEF DESCRIPTION OF THE DRAWINGS

The disclosure may be more completely understood in consideration of the following detailed description in connection with the accompanying drawings, in which:

FIG. 1 illustrates selected aspects of a replacement heart valve implant;

FIG. 2 illustrates selected aspects of an apparatus for compressing the replacement heart valve implant of FIG. 1;

FIG. 3 is a cross-sectional view illustrating selected aspects of the apparatus of FIG. 2;

FIG. 4 is an end view illustrating selected aspects of the apparatus of FIG. 2;

FIG. 5 is an exploded view illustrating selected aspects of the apparatus of FIG. 2;

FIG. 6 is an exploded view illustrating selected aspects of a replacement heart valve system; and

FIGS. 7-10 illustrate aspects of a method of compressing the replacement heart valve implant.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale, wherein like reference numerals indicate like elements throughout the several views. The detailed description and drawings are intended to illustrate but not limit the disclosure. Those skilled in the art will recognize that the various elements described and/or shown may be arranged in various combinations and configurations without departing from the scope of the disclosure. The detailed description and drawings illustrate example embodiments of the disclosure. However, in the interest of clarity and ease of understanding, every feature and/or element may not be shown in each drawing.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The term “extent” may be understood to mean the greatest measurement of a stated or identified dimension, unless the extent or dimension in question is preceded by or identified as a “minimum”, which may be understood to mean the smallest measurement of the stated or identified dimension. For example, “outer extent” may be understood to mean an outer dimension, “radial extent” may be understood to mean a radial dimension, “longitudinal extent” may be understood to mean a longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered the greatest possible dimension measured according to the intended usage, while a “minimum extent” may be considered the smallest possible dimension measured according to the intended usage. In some instances, an “extent” may generally be measured orthogonally within a plane and/or cross-section, but may be, as will be apparent from the particular context, measured differently—such as, but not limited to, angularly, radially, circumferentially (e.g., along an arc), etc.

The terms “monolithic” and “unitary” shall generally refer to an element or elements made from or consisting of a single structure or base unit/element. A monolithic and/or unitary element shall exclude structure and/or features made by assembling or otherwise joining multiple discrete structures or elements together.

The terms “transaortic valve implantation” and “transcatheter aortic valve implantation” may be used interchangeably and may each be referred to using the acronym “TAVI”. The terms “transaortic valve replacement” and “transcatheter aortic valve replacement” may be used interchangeably and may each be referred to using the acronym “TAVR”.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to effect the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

Diseases and/or medical conditions that impact the cardiovascular system are prevalent throughout the world. Traditionally, treatment of the cardiovascular system was often conducted by directly accessing the impacted part of the system. For example, treatment of a blockage in one or more of the coronary arteries was traditionally treated using coronary artery bypass surgery. As can be readily appreciated, such therapies are rather invasive to the patient and require significant recovery times and/or treatments. More recently, less invasive therapies have been developed, for example, where a blocked coronary artery could be accessed and treated via a percutaneous catheter (e.g., angioplasty). Such therapies have gained wide acceptance among patients and clinicians.

Some mammalian hearts (e.g., human, etc.) include four heart valves: a tricuspid valve, a pulmonary valve, an aortic valve, and a mitral valve. Some relatively common medical conditions may include or be the result of inefficiency, ineffectiveness, or complete failure of one or more of the valves within the heart. For example, failure of the aortic valve or the mitral valve can have a serious effect on a human and could lead to a serious health condition and/or death if not dealt with properly. Treatment of defective heart valves poses other challenges in that the treatment often requires the repair or outright replacement of the defective heart valve. Such therapies may be highly invasive to the patient. Disclosed herein is an apparatus, system, and/or method that may be used for preparing and/or delivering a medical implant to a portion of the cardiovascular system in order to diagnose, treat, and/or repair the system. In some embodiments, the apparatus, system, and/or method disclosed herein may be used before and/or during a procedure to diagnose, treat, and/or repair a defective heart valve (e.g., the aortic valve, the mitral valve, etc.). In addition, a replacement heart valve implant may be delivered percutaneously and thus may be much less invasive to the patient. The apparatus, system, and/or method disclosed herein may also provide other desirable features and/or benefits as described below.

It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For example, a reference to “the leaflet”, “the lumen”, or other features may be equally referred to all instances and quantities beyond one of said feature. As such, it will be understood that the following discussion may apply equally to any and/or all of the components for which there are more than one within the replacement heart valve implant and/or the apparatus unless explicitly stated to the contrary.

Additionally, it should be noted that in any given figure, some features may not be shown, or may be shown schematically, for clarity and/or simplicity. Additional details regarding some components and/or method steps may be illustrated in other figures in greater detail. An apparatus and/or system may be used to prepare and/or deliver a variety of medical devices to a number of locations within the anatomy. In at least some embodiments, the apparatus and/or system can be used to prepare and/or deliver a replacement aortic heart valve, and may be discussed herein in that context for brevity. This, however, is not intended to be limiting as the apparatus and/or system may also be used for other interventions including mitral valve replacement, valve repair, and the like, or other similar interventions.

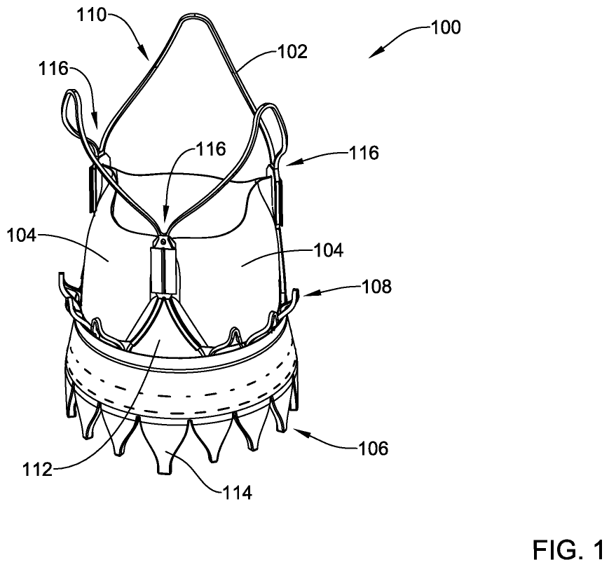

FIG. 1 illustrates selected aspects of a replacement heart valve implant 100. It should be appreciated that the replacement heart valve implant 100 can be any type of heart valve (e.g., a mitral valve, an aortic valve, etc.). In use, the replacement heart valve implant 100 can be implanted (e.g., surgically or through transcatheter delivery) in a mammalian heart. The replacement heart valve implant 100 can be configured to allow one-way flow through the replacement heart valve implant 100 from an inflow end to an outflow end.

The replacement heart valve implant 100 may include an expandable framework 102 defining a central lumen which, in some embodiments, may be substantially cylindrical. The side of the expandable framework 102 and other components facing the central lumen can be referred to as the luminal surface or luminal side. The opposite side of the expandable framework 102 and other components (e.g., facing away from the central lumen) can be referred to as the abluminal surface or abluminal side. In some embodiments, the expandable framework 102 may have a substantially circular cross-section. In some embodiments, the expandable framework 102 can have a non-circular (e.g., D-shaped, elliptical, etc.) cross-section. In some embodiments, a non-circular expandable framework can be used to repair a mitral valve or another non-circular valve in the body. Some suitable but non-limiting examples of materials that may be used to form the expandable framework 102, including but not limited to metals and metal alloys, composites, ceramics, polymers, and the like, are described below.

The expandable framework 102 may include a plurality of frame struts. The frame struts may define a framework or lattice structure. In some embodiments, the expandable framework 102 may define open spaces or interstices between the frame struts. However, in other embodiments the expandable framework 102 may not include such open spaces.

In some embodiments, the expandable framework 102 and/or the frame struts may define a lower crown 106, an upper crown 108, and a plurality of stabilization arches 110. In some embodiments, the lower crown 106 may be disposed at and/or may correspond to the inflow end of the expandable framework 102 and/or the replacement heart valve implant 100. In some embodiments, the upper crown 108 and/or the plurality of stabilization arches 110 may be disposed at and/or may correspond to the outflow end of the expandable framework 102 and/or the replacement heart valve implant 100.

The replacement heart valve implant 100 may include a plurality of leaflets 104 disposed within the central lumen. Each of the plurality of leaflets 104 may include a root edge coupled to the expandable framework 102 and a free edge (e.g., a coaptation edge) movable relative to the root edge to coapt with the coaptation edges of the other leaflets along a coaptation region. In some embodiments, the plurality of leaflets 104 can be integrally formed with each other, such that the plurality of leaflets 104 is formed as a single unitary and/or monolithic unit. In some embodiments, a “root edge” can be a formed edge, such as when the plurality of leaflets 104 is formed in place on the expandable framework 102. In some embodiments, the plurality of leaflets 104 may be formed integrally with other structures such as an inner skirt 112 and/or an outer skirt 114, base structures, liners, or the like and in those circumstances the “root edge” is not a cut or otherwise divided edge, but rather is the location opposite the free edge where each of the plurality of leaflets 104 meets those other structures.

The free edges of the plurality of leaflets 104 may move into coaptation with one another in a closed position to substantially restrict fluid from flowing past the replacement heart valve implant 100. Specifically, the plurality of leaflets 104 may coapt to fill up or close the central lumen of the replacement heart valve implant 100 thereby impeding the flow of fluid in an upstream or retrograde direction. The free edges of the plurality of leaflets 104 may be move apart from each other in an open position to permit fluid flow through the replacement heart valve implant 100. Specifically, the plurality of leaflets 104 may move apart from each other to open the central lumen of the replacement heart valve implant 100 thereby permitting the flow of fluid in a downstream or antegrade direction. In FIG. 1, the plurality of leaflets 104 is shown in the open position or an a partially open position (e.g., a neutral position) that the plurality of leaflets 104 may move to when unbiased by fluid flow.

Each of the plurality of leaflets 104 may further include two connection portions. One connection portion can be disposed on either end of the free edge of its respective leaflet such that the connection portions are contacting or adjacent to the expandable framework 102 at a plurality of commissures 116. In some embodiments, the plurality of leaflets 104 may be secured and/or attached to the expandable framework 102 at the plurality of commissures 116. The free edges of the plurality of leaflets 104 may extend between the plurality of commissures 116.

In some embodiments, the plurality of commissures 116 may be disposed at a base of the plurality of stabilization arches 110. In some embodiments, each of the plurality of commissures 116 may join circumferentially adjacent stabilization arches of the plurality of stabilization arches 110 together. In some embodiments, the plurality of commissures 116 may be disposed distal of the plurality of stabilization arches 110 and proximal of the upper crown 108. In at least some embodiments, between circumferentially adjacent commissures of the plurality of commissures 116, the replacement heart valve implant 100 may be devoid of the expandable framework 102 at a longitudinal position radially outward of the free edges of the plurality of leaflets 104. As such, the free edges of the plurality of leaflets 104 may be free from direct contact with the expandable framework 102 as the plurality of leaflets 104 open and/or close.

In some embodiments, the connections portions of the plurality of leaflets 104 may also be referred to as commissural mounting tabs. In some embodiments, the connection portions can be disposed at least partially within a connection aperture defined and/or extending through the expandable framework 102 thereby coupling or attaching the plurality of leaflets 104 to the expandable framework 102. In some embodiments, the connection portions can be projections from their respective leaflet. In some embodiments, the connection portions may be integrally formed with its respective leaflet, such that the leaflet and connection portions are a single unitary and/or monolithic part or structure. In some embodiments, the connection portions of the leaflet can extend completely through the connection apertures, such as when the connection apertures extend completely through the expandable framework 102.

In some embodiments, the connection portions may encircle a portion of the expandable framework 102, such as when the connection portion contacts a strut at a location where the strut and/or the expandable framework 102 does not define a connection aperture. In some embodiments, the plurality of leaflets 104 and/or the connection portions may be attached to the expandable framework 102 using sutures, adhesives, or other suitable methods.

In some embodiments, the replacement heart valve implant 100 may include the inner skirt 112. In some embodiments, the inner skirt 112 may define a substantially tubular shape. The inner skirt 112 may be disposed on the luminal surface of the expandable framework 102. The inner skirt 112 may direct fluid, such as blood, flowing through the replacement heart valve implant 100 toward the plurality of leaflets 104. The inner skirt 112 may ensure the fluid flows through the central lumen of the replacement heart valve implant 100 and does not flow around the plurality of leaflets 104 when they are in the closed position.

The inner skirt 112 may include a connection projection that extends from the inner skirt 112 and into one or more connection aperture. In some embodiments, the connection projection may extend around a portion of a strut and/or the expandable framework 102. In some embodiments, the connection projection may extend around a portion of a strut and into one or more connection aperture. In some embodiments, the connection projections may interact with the expandable framework 102 to attach or couple the inner skirt 112 to the expandable framework 102 through surface area contact and/or a form fitting configuration. In some embodiments, the connection projections may be attached to the expandable framework 102 using sutures, adhesives, or other suitable methods.

In some embodiments, the replacement heart valve implant 100 can include the outer skirt 114. In some embodiments, the outer skirt 114 may define a substantially tubular shape. In some embodiments, the outer skirt 114 may be disposed on the abluminal surface of the expandable framework 102. In some embodiments, the outer skirt 114 can be disposed between the expandable framework 102 and the vessel wall in order to prevent fluid, such as blood, flowing around the replacement heart valve implant 100 and/or the expandable framework 102. The outer skirt 114 may ensure the fluid flows through the replacement heart valve implant 100 and does not flow around the replacement heart valve implant 100, such as to ensure that the plurality of leaflets 104 can stop the flow of fluid when in the closed position.

The outer skirt 114 may include a connection projection that extends from the outer skirt 114 and into one or more connection aperture. In some embodiments, the connection projection may extend around a portion of a strut and/or the expandable framework 102. In some embodiments, the connection projection may extend around a portion of a strut and/or the expandable framework 102 and into one or more connection aperture. In some embodiments, the connection projections may interact with the expandable framework 102 to attach or couple the outer skirt 114 to the expandable framework 102, such as through surface area contact or a form fitting configuration. In some embodiments, the connection projections may be attached to the expandable framework 102 using sutures, adhesives, or other suitable methods.

In some embodiments, the plurality of leaflets 104 may be comprised of a polymer, such as a thermoplastic polymer. In some embodiments, the plurality of leaflets 104 may include at least 50 percent by weight of a polymer. In some embodiments, the plurality of leaflets 104 may be formed from bovine pericardial or other living tissue. Other configurations and/or materials are also contemplated.

In some embodiments, the inner skirt 112 may include a polymer, such as a thermoplastic polymer. In some embodiments, the inner skirt 112 may include at least 50 percent by weight of a polymer. In some embodiments, the outer skirt 114 may include a polymer, such as a thermoplastic polymer. In some embodiments, the outer skirt 114 may include at least 50 percent by weight of a polymer. In some embodiments one or more of the plurality of leaflets 104, the inner skirt 112, and/or the outer skirt 114 may be formed of the same polymer or polymers. In some embodiments, the polymer may be a polyurethane. Some suitable but non-limiting examples of materials that may be used to form the inner skirt 112 and/or the outer skirt 114, including but not limited to polymers, composites, and the like, are described below.

In some embodiments, the inner skirt 112 may be coupled to the lower crown 106 and/or the upper crown 108. In some embodiments, the inner skirt 112 may be coupled only to the upper crown 108. In some embodiments, the outer skirt 114 may be coupled to the lower crown 106 and/or the upper crown 108. In some embodiments, the outer skirt 114 may be coupled only to the lower crown 106. In some embodiments, the plurality of leaflets 104 may be coupled to the expandable framework 102 at a position that is at or just below the plurality of stabilization arches 110 and above the upper crown 108.

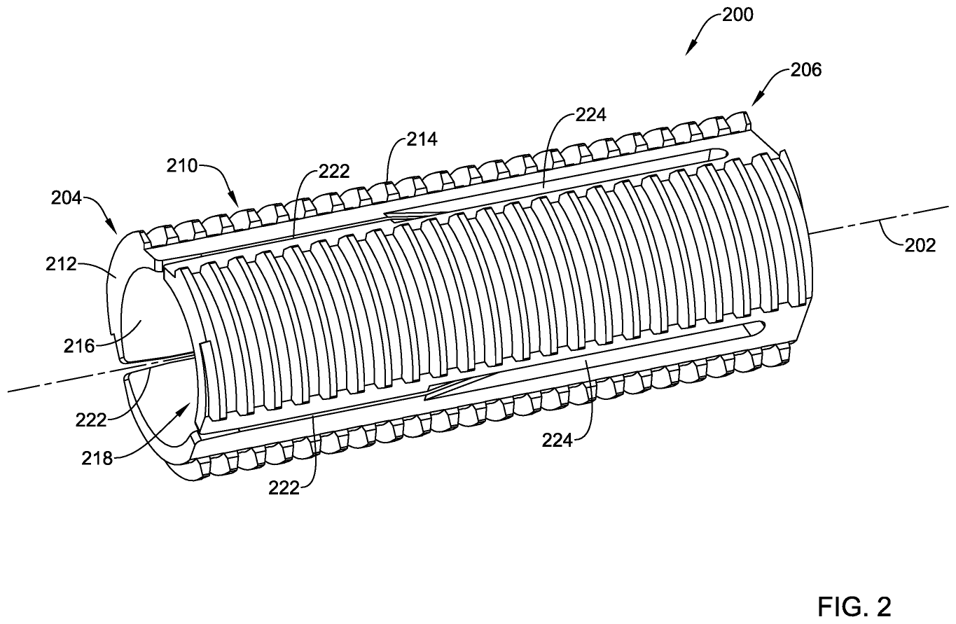

FIGS. 2-4 illustrate selected aspects of an apparatus 200 for compressing the replacement heart valve implant 100. The apparatus 200 may include a tubular member 210 having a wall 212 having an outer surface 214 and an inner surface 216 defining a lumen 218 of the tubular member 210, as seen in FIG. 2, extending from a proximal end 204 of the tubular member 210 to a distal end 206 of the tubular member 210. In some embodiments, the apparatus 200 and/or the tubular member 210 may include external threads formed in the outer surface 214 of the tubular member 210. Some suitable but non-limiting examples of materials that may be used to form the tubular member 210, including but not limited to metals and metal alloys, composites, ceramics, polymers, and the like, are described below. In some embodiments, the tubular member 210 may be formed from a polymeric material.

In some embodiments, the apparatus 200 and/or the tubular member 210 may include a plurality of longitudinal slots 222 formed in and/or extending through the wall 212 of the tubular member 210 from the outer surface 214 to the inner surface 216. In some embodiments, the plurality of longitudinal slots 222 may extend from the proximal end 204 of the tubular member 210 toward the distal end 206 of the tubular member 210. In some embodiments, the plurality of longitudinal slots 222 may begin at and/or extend from a proximal-most surface of the tubular member 210 toward the distal end 206 of the tubular member 210. In some embodiments, the apparatus 200 and/or the tubular member 210 may include a plurality of recesses 224 formed in the wall 212 and/or the outer surface 214 of the tubular member 210. In at least some embodiments, the plurality of recesses 224 may taper radially inwardly in a distal direction. For example, a distal end of the plurality of recesses 224 may extend radially inward a greater distance from the outer surface 214 than a proximal end of the plurality of recesses 224. In some embodiments, the proximal end of the plurality of recesses 224 may be distal of the proximal end 204 of the tubular member 210. In some embodiments, the proximal end of the plurality of recesses 224 may be disposed within a medial region of the tubular member 210, and the distal end of the plurality of recesses 224 may be disposed within a distal region of the tubular member 210. Other configurations are also contemplated.

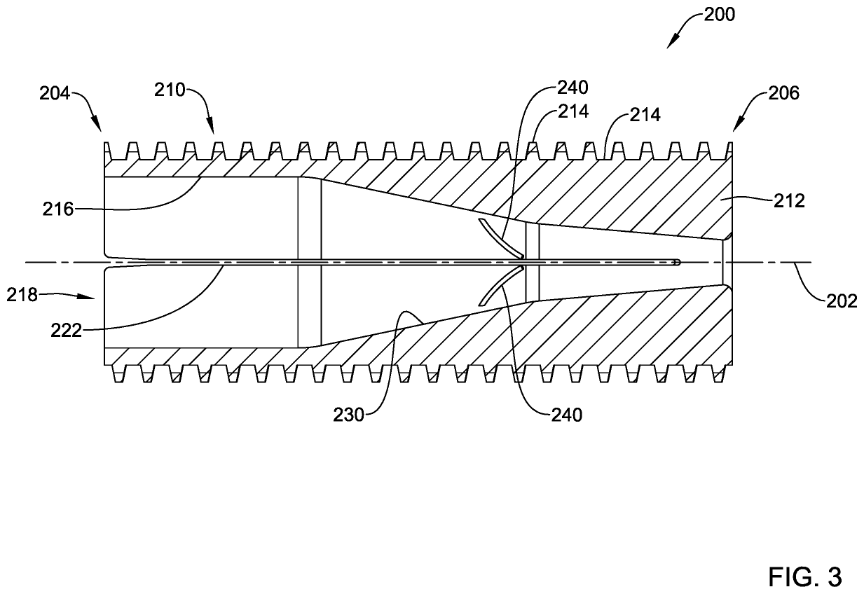

In at least some embodiments, the inner surface 216 of the tubular member 210 may include a tapered portion 230 having a radially inward taper in a distal direction, as seen in the cross-sectional view of FIG. 3. The apparatus 200 may include a plurality of flexible arms 240 projecting radially inward from the tapered portion 230 of the inner surface 216 of the tubular member 210. In some embodiments, the plurality of flexible arms 240 may include two flexible arms, three flexible arms, four flexible arms, or another suitable number of flexible arms. In some embodiments, the number of flexible arms may be a multiple of the number of leaflets in the plurality of leaflets 104. For example, if the plurality of leaflets 104 includes three leaflets, the plurality of flexible arms 240 may include three flexible arms, six flexible arms, etc. Other configurations are also contemplated. Some suitable but non-limiting examples of materials that may be used to form the plurality of flexible arms 240, including but not limited to metals and metal alloys, composites, ceramics, polymers, and the like, are described below. In some embodiments, the plurality of flexible arms 240 may be formed from a polymeric material. In some embodiments, the plurality of flexible arms 240 may be formed from a monofilament polymeric material. In some embodiments, the plurality of flexible arms 240 may be formed from a different material than the tubular member 210. Other configurations are also contemplated.

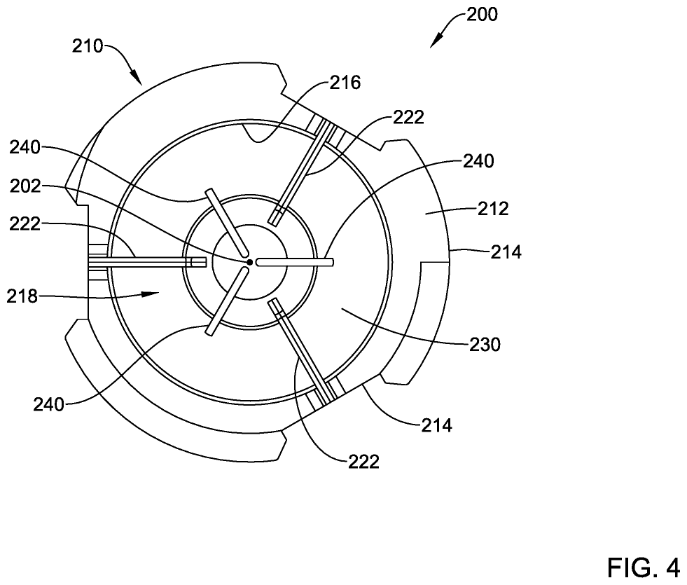

In some embodiments, the plurality of flexible arms 240 may be spaced apart circumferentially around the central longitudinal axis 202 of the tubular member 210. In some embodiments, the plurality of flexible arms 240 may be equally spaced apart circumferentially around the central longitudinal axis 202 of the tubular member 210. In some embodiments, the plurality of flexible arms 240 may be spaced apart about 120 degrees from each other, as seen in FIG. 4 for example. Other configurations and/or spacing are also contemplated, such as equidistant spacing and non-equidistant spacing. In some embodiments, each of the plurality of flexible arms 240 may be disposed circumferentially between two circumferentially adjacent longitudinal slots of the plurality of longitudinal slots 222. As will become apparent, in some embodiments, quantity of flexible arms and/or spacing between adjacent flexible arms of the plurality of flexible arms 240, as well as other elements, structures, and/or features, may be related to and/or dependent on the number of leaflets in the plurality of leaflets 104.

The plurality of flexible arms 240 may be attached and/or secured to the wall 212 of the tubular member 210. In some embodiments, the plurality of flexible arms 240 may be fixedly attached to the wall 212 of the tubular member 210. For example, the plurality of flexible arms 240 may be adhesively bonded to the wall 212 of the tubular member 210, or the plurality of flexible arms 240 may be welded to the wall 212 of the tubular member 210. Other configurations are also contemplated.

In some embodiments, the plurality of flexible arms 240 may project radially inward from the inner surface 216 of the tubular member 210 at least 50% of a distance from the inner surface 216 of the tubular member 210, at a position where the plurality of flexible arms 240 is attached and/or secured to the tubular member 210, to the central longitudinal axis 202 of the tubular member 210 in an unbiased configuration. In some embodiments, the plurality of flexible arms 240 may project radially inward from the inner surface 216 of the tubular member 210 at least 75% of the distance from the inner surface 216 of the tubular member 210, at the position where the plurality of flexible arms 240 is attached and/or secured to the tubular member 210, to the central longitudinal axis 202 of the tubular member 210 in the unbiased configuration. In some embodiments, the plurality of flexible arms 240 may project radially inward from the inner surface 216 of the tubular member 210 at least 80% of the distance from the inner surface 216 of the tubular member 210, at the position where the plurality of flexible arms 240 is attached and/or secured to the tubular member 210, to the central longitudinal axis 202 of the tubular member 210 in the unbiased configuration. In some embodiments, the plurality of flexible arms 240 may project radially inward from the inner surface 216 of the tubular member 210 at least 85% of the distance from the inner surface 216 of the tubular member 210, at the position where the plurality of flexible arms 240 is attached and/or secured to the tubular member 210, to the central longitudinal axis 202 of the tubular member 210 in the unbiased configuration. In some embodiments, the plurality of flexible arms 240 may project radially inward from the inner surface 216 of the tubular member 210 at least 90% of the distance from the inner surface 216 of the tubular member 210, at the position where the plurality of flexible arms 240 is attached and/or secured to the tubular member 210, to the central longitudinal axis 202 of the tubular member 210 in the unbiased configuration.

In some embodiments, the plurality of flexible arms 240 may extend from the inner surface 216 of the tubular member 210 in the distal direction within the lumen 218 of the tubular member 210 in the unbiased configuration. This may be most clearly seen in FIG. 3. However, other configurations are also contemplated. For example, in some embodiments, the plurality of flexible arms 240 may extend radially inward from the inner surface 216 at a right angle to the central longitudinal axis 202 of the tubular member 210 in the unbiased configuration. In some embodiments, the plurality of flexible arms 240 may be curved and/or may form an arc in the unbiased configuration. In some embodiments, the plurality of flexible arms 240 may be substantially straight in the unbiased configuration.

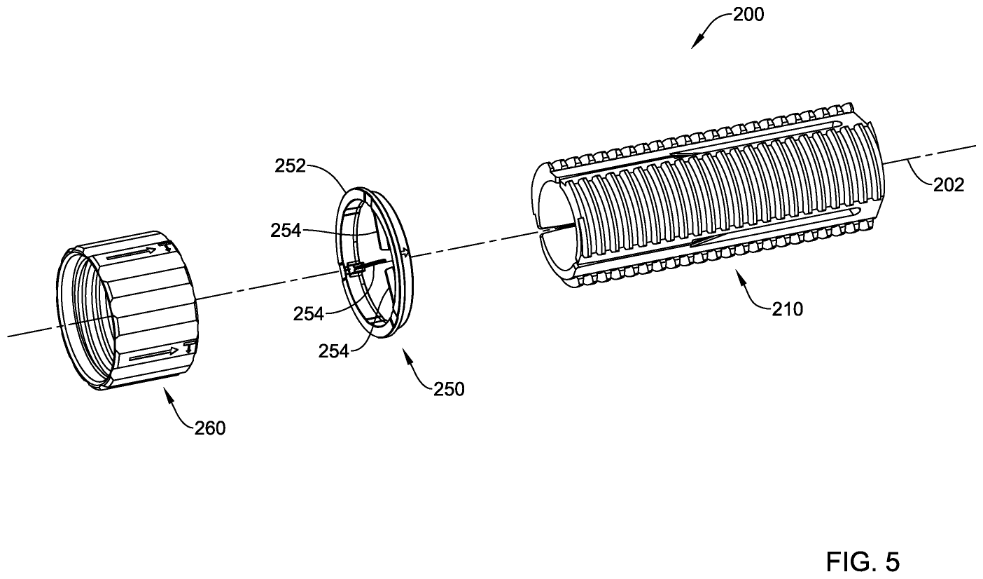

As illustrated in FIG. 5, the apparatus 200 for compressing the replacement heart valve implant 100 may further include a push member 250 comprising an annular ring 252 and a plurality of extension members 254 extending radially inward from the annular ring 252. In at least some embodiments, the plurality of extension members 254 may extend radially inward to free ends. In some embodiments, the free ends of the plurality of extension members 254 may be spaced apart from each other about a center and/or a central axis of the annular ring 252. The push member 250 and/or the annular ring 252 may be configured to slide over the outer surface 214 of the tubular member 210 and/or the external threads of the tubular member 210. In some embodiments, the plurality of extension members 254 may extend through the plurality of longitudinal slots 222 into the lumen 218 of the tubular member 210 as the push member 250 and/or the annular ring 252 slides over the outer surface 214 of the tubular member 210 and/or the external threads of the tubular member 210. Some suitable but non-limiting examples of materials that may be used to form the push member 250, including but not limited to metals and metal alloys, composites, ceramics, polymers, and the like, are described below. In some embodiments, the push member 250 may be formed from a polymeric material.

The apparatus 200 for compressing the replacement heart valve implant 100 may further include a threaded nut 260 having internal threads configured to engage the external threads formed in the outer surface 214 of the tubular member 210. The threaded nut 260 may be configured to rotate around the tubular member 210. Rotation of the threaded but 260 around the tubular member 210 may translate and/or advance the threaded but 260 distally along the outer surface 214 of the tubular member 210. Some suitable but non-limiting examples of materials that may be used to form the threaded nut 260, including but not limited to metals and metal alloys, composites, ceramics, polymers, and the like, are described below. In some embodiments, the threaded nut 260 may be formed from a polymeric material.

In some embodiments, the push member 250 and the threaded nut 260 may be formed from the same material. In some embodiments, the push member 250 and the threaded nut 260 may be formed from different materials. In some embodiments, the push member 250 and/or the threaded nut 260 may be formed from the same material as the tubular member 210. In some embodiments, the push member 250 and/or the threaded nut 260 may be formed from a different material than the tubular member 210.

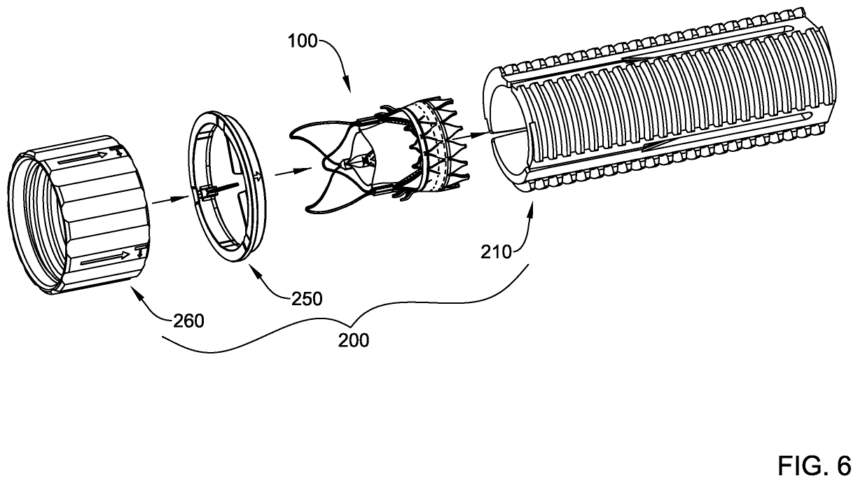

FIG. 6 is an exploded view illustrating aspects of a replacement heart valve system. In at least some embodiments, the replacement heart valve system may include the replacement heart valve implant 100 and the apparatus 200 for compressing the replacement heart valve implant 100. The replacement heart valve implant 100 may be inserted into the apparatus 200 as shown.

The system of FIG. 6 may be used in a method of compressing the replacement heart valve implant 100. The method may include inserting the inflow end of the expandable framework 102 and/or the replacement heart valve implant 100 into the lumen 218 of the tubular member 210 of the apparatus 200 for compressing the replacement heart valve implant 100, as generally shown in FIG. 6. The method may include sliding the push member 250 over the proximal end 204 of the tubular member 210 until the plurality of extension members 254 engage the plurality of commissures 116 of the replacement heart valve implant 100. As the push member 250 and/or the annular ring 252 is engaged with and/or advanced over the tubular member 210, the plurality of stabilization arches 110 may be positioned between the plurality of extension members 254 such that the plurality of extension members 254 align with the plurality of commissures 116. The plurality of extension members 254 may be advanced into and/or may extend through the plurality of longitudinal slots 222 as the annular ring 252 slides over the outer surface 214 of the tubular member 210.

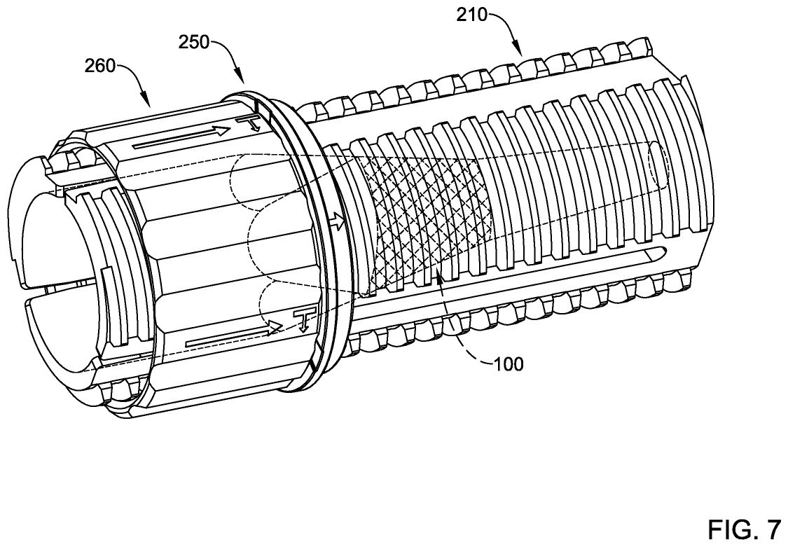

The push member 250 may be configured to push the replacement heart valve implant 100 through the lumen 218 of the tubular member 210. The plurality of extension members 254 may be configured to engage and/or push against the plurality of commissures 116 of the replacement heart valve implant 100 when the push member 250 pushes the replacement heart valve implant 100 through the tubular member 210. As the replacement heart valve implant 100 is pushed and/or advanced distally through the lumen 218 of the tubular member 210, the inner surface 216 and/or the tapered portion 230 of the tubular member 210 gradually compresses the expandable framework 102 and/or the replacement heart valve implant 100 radially inward, as shown in FIG. 7, toward a delivery configuration.

The method may include placing the threaded nut 260 over the proximal end 204 of the tubular member 210. As discussed herein, the threaded nut 260 may include internal threads configured to engage the external threads formed in the outer surface 214 of the tubular member 210. The threaded nut 260 may be configured to rotate around the tubular member 210 and doing so may advance the threaded nut 260 toward the distal end 206 of the tubular member 210. In some embodiments, clockwise rotation of the threaded nut 260 around the tubular member 210, as viewed proximally to distally, may advance the threaded nut 260 distally along the tubular member 210. A distal surface of the threaded nut 260 may be configured to engage a proximal surface of the push member 250 and/or the annular ring 252.

The method may include advancing the threaded nut 260 distally along and/or over the tubular member 210 to translate the push member 250 distally along the tubular member 210, the push member 250 advancing the replacement heart valve implant 100 distally within the lumen 218 of the tubular member 210. Advancing the replacement heart valve implant 100 distally within the lumen 218 and/or the tapered portion 230 of the tubular member 210 may radially compress the expandable framework 102 and/or the replacement heart valve implant 100.

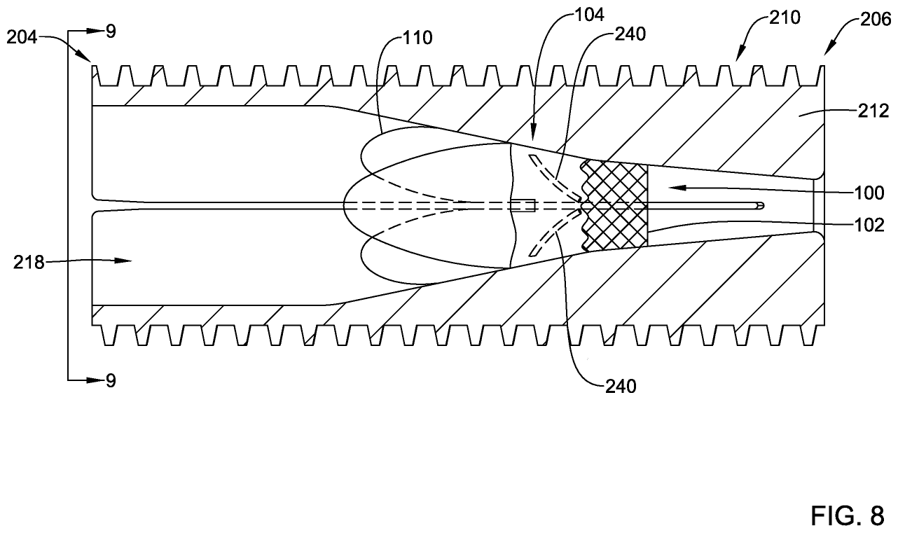

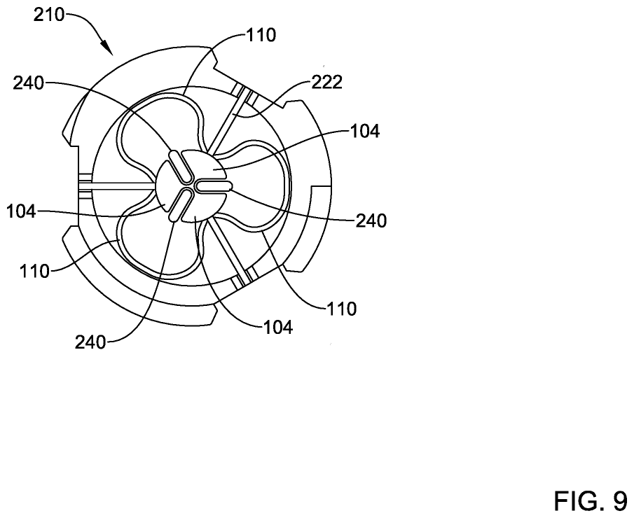

As the replacement heart valve implant 100 advances through the lumen 218 and/or the tapered portion 230 of the tubular member 210 past the plurality of flexible arms 240, the plurality of flexible arms 240 may be configured to push the plurality of leaflets 104 of the replacement heart valve implant 100 radially inward from the expandable framework 102 and/or the plurality of commissures 116 of the replacement heart valve implant 100 as the replacement heart valve implant 100 passes through the tubular member 210, as seen in FIGS. 8 and 9. For improved clarity, the push member 250 and the threaded nut 260 are not illustrated in FIGS. 8 and 9, and FIG. 8 is shown in partial cross-section while FIG. 9 is an end view of the system in the position shown in FIG. 8.

As may be seen in FIG. 9, each of the plurality of flexible arms 240 may be disposed circumferentially between two circumferentially adjacent longitudinal slots of the plurality of longitudinal slots 222. Accordingly, since the plurality of extension members 254 of the push member 250 extends through the plurality of longitudinal slots 222, each of the plurality of flexible arms 240 may be disposed circumferentially between two circumferentially adjacent extension members of the plurality of extension members 254. The plurality of leaflets 104 and/or the free edges of the plurality of leaflets 104 may be aligned with the plurality of flexible arms 240. As the upper crown 108 moves past the plurality of flexible arms 240, the plurality of flexible arms 240 may engage the plurality of leaflets 104 as the replacement heart valve implant 100 is advanced distally within the lumen 218 of the tubular member 210.

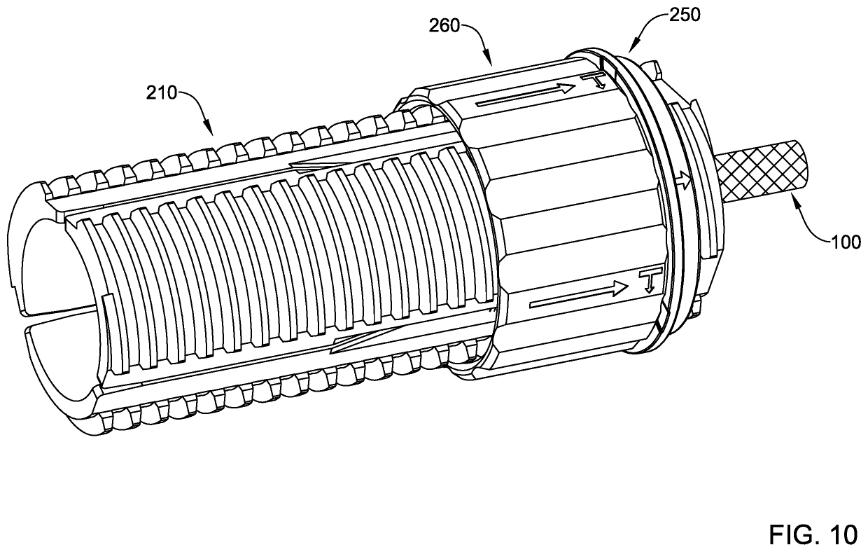

In at least some embodiments, each of the plurality of leaflets 104 may be engaged by only one of the plurality of flexible arms 240 as the replacement heart valve implant 100 passes through the lumen 218 of the tubular member 210, as shown in FIG. 9. In some embodiments, each of the plurality of leaflets 104 may be engaged by more than one of the plurality of flexible arms 240, such as in embodiments having a number of flexible arms that is a multiple of the number of leaflets (e.g., three leaflets and six flexible arms, etc.). Other configurations are also contemplated. As seen in FIG. 9, the plurality of flexible arms 240 may be configured to fold or aid in folding the plurality of leaflets 104 radially inward as the expandable framework 102 and/or the replacement heart valve implant 100 is radially compressed toward the delivery configuration. As the inflow end of the replacement heart valve implant 100 exits the distal end 206 of the tubular member 210, the expandable framework 102 and/or the replacement heart valve implant 100 may be in the delivery configuration, as seen in FIG. 10.

In some embodiments, the expandable framework 102 and/or the replacement heart valve implant 100 may be radially compressed from an outer diameter of about 23 millimeters (mm), about 25 mm, about 27 mm, about 30 mm, etc. in an unconstrained configuration to about 10 mm, about 9 mm about 8 mm, about 7 mm, about 6 mm, etc. in the delivery configuration. Other configurations are also contemplated.

The materials that can be used for the various components of the apparatus and/or system and the various elements thereof disclosed herein may include those commonly associated with medical devices. For simplicity purposes, the following discussion refers to the system. However, this is not intended to limit the devices and methods described herein, as the discussion may be applied to other elements, members, components, or devices disclosed herein, such as, but not limited to, the expandable framework, the inner skirt, the outer skirt, the plurality of leaflets, the tubular member, the push member, the threaded nut, the plurality of flexible arms, and/or elements or components thereof.

In some embodiments, the system and/or components thereof may be made from a metal, metal alloy, polymer (some examples of which are disclosed below), a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, polyisobutylene (PIB), polyisobutylene polyurethane (PIBU), polyurethane silicone copolymers (for example, Elast-Eon® from AorTech Biomaterials or ChronoSil® from AdvanSource Biomaterials), ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In at least some embodiments, portions or all of the system and/or components thereof may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the system in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the system to achieve the same result.

In some embodiments, a degree of Magnetic Resonance Imaging (MRI) compatibility is imparted into the system and/or other elements disclosed herein. For example, the system and/or components or portions thereof, may be made of a material that does not substantially distort the image and create substantial artifacts (i.e., gaps in the image). Certain ferromagnetic materials, for example, may not be suitable because they may create artifacts in an MRI image. The system or portions thereof may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

In some embodiments, the system and/or other elements disclosed herein may include a fabric material disposed over or within the structure. The fabric material may be composed of a biocompatible material, such a polymeric material or biomaterial, adapted to promote tissue ingrowth. In some embodiments, the fabric material may include a bioabsorbable material. Some examples of suitable fabric materials include, but are not limited to, polyethylene glycol (PEG), nylon, polytetrafluoroethylene (PTFE, ePTFE), a polyolefinic material such as a polyethylene, a polypropylene, polyester, polyurethane, and/or blends or combinations thereof.

In some embodiments, the system and/or other elements disclosed herein may include and/or be formed from a textile material. Some examples of suitable textile materials may include synthetic yarns that may be flat, shaped, twisted, textured, pre-shrunk or un-shrunk. Synthetic biocompatible yarns suitable for use in the present disclosure include, but are not limited to, polyesters, including polyethylene terephthalate (PET) polyesters, polypropylenes, polyethylenes, polyurethanes, polyolefins, polyvinyls, polymethylacetates, polyamides, naphthalene dicarboxylene derivatives, natural silk, and polytetrafluoroethylenes. Moreover, at least one of the synthetic yarns may be a metallic yarn or a glass or ceramic yarn or fiber. Useful metallic yarns include those yarns made from or containing stainless steel, platinum, gold, titanium, tantalum or a Ni—Co−Cr-based alloy. The yarns may further include carbon, glass or ceramic fibers. Desirably, the yarns are made from thermoplastic materials including, but not limited to, polyesters, polypropylenes, polyethylenes, polyurethanes, polynaphthalenes, polytetrafluoroethylenes, and the like. The yarns may be of the multifilament, monofilament, or spun types. The type and denier of the yarn chosen may be selected in a manner which forms a biocompatible and implantable prosthesis and, more particularly, a vascular structure having desirable properties.

In some embodiments, the system and/or other elements disclosed herein may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethylketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl ketone, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); cholesterol-lowering agents; vasodilating agents; and agents which interfere with endogenous vasoactive mechanisms.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. An apparatus for compressing a replacement heart valve implant, comprising:
a tubular member having a wall having an outer surface and an inner surface defining a lumen of the tubular member, wherein the inner surface includes a tapered portion having a radially inward taper in a distal direction; and
a plurality of flexible arms projecting radially inward from the tapered portion;
wherein the plurality of flexible arms projects radially inward from the inner surface at least 50% of a distance from the inner surface to a central longitudinal axis of the tubular member in an unbiased configuration.

2. The apparatus of claim 1, wherein the plurality of flexible arms projects radially inward from the inner surface at least 75% of the distance from the inner surface to the central longitudinal axis of the tubular member in the unbiased configuration.

3. The apparatus of claim 1, wherein the plurality of flexible arms is fixedly attached to the wall of the tubular member.

4. The apparatus of claim 1, wherein the plurality of flexible arms is formed from a monofilament polymeric material.

5. The apparatus of claim 1, wherein the plurality of flexible arms is formed from a different material then the tubular member.

6. The apparatus of claim 1, further comprising:
a threaded nut configured to engage threads formed in the outer surface of the tubular member; and
a push member comprising an annular ring and a plurality of extension members extending radially inward from the annular ring;
wherein the annular ring is configured to slide over the outer surface of the tubular member.

7. The apparatus of claim 6, wherein the tubular member includes a plurality of longitudinal slots extending through the wall;
wherein the plurality of extension members extends through the plurality of longitudinal slots into the lumen of the tubular member as the annular ring slides over the outer surface of the tubular member.

8. A replacement heart valve system, comprising:
a replacement heart valve implant including an expandable framework and a plurality of leaflets secured to the expandable framework; and
an apparatus for compressing the replacement heart valve implant comprising:
a tubular member having a wall having an outer surface and an inner surface defining a lumen of the tubular member, wherein the inner surface includes a tapered portion having a radially inward taper in a distal direction; and
a plurality of flexible arms projecting radially inward from the tapered portion;
wherein the plurality of flexible arms is configured to push the plurality of leaflets radially inward as the replacement heart valve implant passes through the tubular member.

9. The replacement heart valve system of claim 8, wherein the plurality of flexible arms is equally spaced apart circumferentially around a central longitudinal axis of the tubular member.

10. The replacement heart valve system of claim 9, wherein the plurality of flexible arms is spaced apart about 120 degrees from each other.

11. The replacement heart valve system of claim 8, wherein each of the plurality of leaflets is engaged by only one of the plurality of flexible arms as the replacement heart valve implant passes through the tubular member.

12. The replacement heart valve system of claim 8, wherein the plurality of leaflets is secured to the expandable framework at a plurality of commissures and includes free edges extending between the plurality of commissures, and wherein between circumferentially adjacent commissures the replacement heart valve implant is devoid of the expandable framework at a longitudinal position radially outward of the free edges of the plurality of leaflets.

13. A method of compressing a replacement heart valve implant, comprising:
inserting an inflow end of the replacement heart valve implant into an apparatus for compressing the replacement heart valve implant, wherein the apparatus comprises:
a tubular member having a wall having an outer surface and an inner surface defining a lumen of the tubular member, wherein the inner surface includes a tapered portion having a radially inward taper in a distal direction; and
a plurality of flexible arms projecting radially inward from the tapered portion;
sliding a push member over a proximal end of the tubular member, the push member comprising an annular ring and a plurality of extension members extending radially inward from the annular ring, until the plurality of extension members engages a plurality of commissures of the replacement heart valve implant;
placing a threaded nut over the proximal end of the tubular member, the threaded nut being configured to engage threads formed in the outer surface of the tubular member;
advancing the threaded nut distally along the tubular member to translate the push member distally along the tubular member, the push member advancing the replacement heart valve implant distally within the lumen of the tubular member.

14. The method of claim 13, wherein as the replacement heart valve implant advances past the plurality of flexible arms, the plurality of flexible arms pushes a plurality of leaflets of the replacement heart valve implant radially inward from an expandable framework of the replacement heart valve implant.

15. The method of claim 13, wherein each of the plurality of flexible arms is disposed circumferentially between two circumferentially adjacent extension members of the plurality of the extension members.

16. The method of claim 13, wherein the plurality of extension members extends radially inward through a plurality of longitudinal slots formed in the tubular member.

17. The method of claim 16, wherein the plurality of longitudinal slots extends from the proximal end of the tubular member toward a distal end of the tubular member.

18. The method of claim 16, wherein each of the plurality of flexible arms is disposed circumferentially between two circumferentially adjacent longitudinal slots of the plurality of the longitudinal slots.

19. The method of claim 13, wherein advancing the replacement heart valve implant distally within the lumen of the tubular member radially compresses the replacement heart valve implant.

20. The method of claim 13, wherein the plurality of flexible arms extends from the inner surface of the tubular member in the distal direction within the lumen of the tubular member.
